(19) United States
(12) Patent Application Publication
(10) Pub. No.: US 2009/0018098 A1
(43) Pub. Date: Jan. 15, 2009

(54) TARGETING THE ABSENCE: HOMOZYGOUS DNA DELETIONS AS SIGNPOSTS FOR CANCER THERAPY

(75) Inventor: Alexander Varshavsky, La Canada, CA (US)

Correspondence Address: KNOBBE MARTENS OLSON & BEAR LLP, 2040 MAIN STREET, FOURTEENTH FLOOR, IRVINE, CA, 92614, US

(73) Assignee: CALIFORNIA INSTITUTE OF TECHNOLOGY, Pasadena, CA (US)

(21) Appl. No.: 12/172,058

(22) Filed: Jul. 11, 2008

Related U.S. Application Data
(60) Provisional application No. 60/959,446, filed on Jul. 13, 2007.

(51) Int. Cl. A61K 31/7088; C12P 21/04; C12Q 1/68; C12N 15/00

(52) U.S. Cl. 514/44; 435/69.1; 435/320.1; 435/6

(57) ABSTRACT

The present application relates to methods and compositions for targeting a homozygous DNA deletion (HD) in cells. Methods, structures, vectors and compositions for activating a payload in cells having an HD are provided. In some embodiments, the method comprises administering, to a population of cells comprising at least one cell having an HD, a nucleic acid vector encoding two complementary protein fusion molecules and a payload such that the payload is selectively activated only in cells having a HD. HDs are attractive “negative” targets for cancer therapy because of their immutability and prevalence in cancer cells. Thus, in some embodiments methods of treating cancer by specifically delivering a payload, such as a toxin, to cancer cells comprising one or more particular HDs are provided.

### CROSS-REFERENCE TO RELATED APPLICATIONS

This application claims priority under 35 U.S.C. §119(e) to U.S. Provisional Application No. 60/959,446 filed Jul. 13, 2007, which is incorporated herein by reference in its entirety.

### STATEMENT REGARDING FEDERALLY SPONSORED R&D

This invention was made with government support under Grant Numbers: GM31530 and DK39520 awarded by the National Institutes of Health. The government has certain rights in the invention.

### BACKGROUND

Studies have demonstrated that many cancers, including major ones, contain a significant number of scattered homozygous deletions (HDs). Recent methods for detecting copy number changes in large genomes added a number of cancer-associated homozygous deletions to the HDs that were unambiguously identified in earlier studies. Many cancers harbor HDs at fragile sites, visualized as chromosomal regions that stain weakly in mitotic chromosome spreads of cells subjected to stress during DNA replication. For example, human cell lines derived from carcinomas of the stomach, lung, breast, ovary and colon frequently contain HDs, of a few to 200 kb in size at one or both of the two fragile sites called FRA3B and FRA16D (see, for example, Finnis et al. (2005) Hum Mol Genet 14, 1341-1349 and references therein). Many other homozygous deletions were also identified in various cancers and in cell lines derived from them. These HDs often encompass specific tumor suppressor genes such as, for example, SMARCB1/INI1 or PTEN, and other regions that are either known or suspected to contain tumor suppressors. (Finnis et al. (2005) Hum Mol Genet 14, 1341-1349; Kost-Alimova et al. (2007) Sem Cancer Biol 17, 19-30; Modena et al. (2005) Cancer Res 65, 4012-4019; Tagawa et al. (2005) Oncogene 24, 1348-1358; Jonsson et al (2007) Genes Chromosomes Cancer 46, 543-558; Sun et al. (2007) Prostate 67, 692-700; Nakaya et al. (2007) Oncogene 26, 1-9; Cox et al. (2005) Proc Natl Acad Sci USA 102, 4542-4547; Struski et al. (2007) Cancer Genet Cytogenet 174, 151-160; Hamaguchi et al. (2002) Proc Natl Acad Sci USA 99, 13647-13652; Hustinx et al. (2005) Cancer Biol Ther 4, 83-86; Kasahara et al (2006) Anticancer Res 26, 4299-4306; Seng et al. (2005) Genes Chromosomes Cancer 43, 181-193; Largo et al. (2007) Haematologica 92, 795-802.) Another example is myeloma cells, which have been shown to exhibit a broad range of copy-number changes in specific DNA regions, including multiple HDs whose sizes varied from 20 kb to 11 Mb (Largo et al. (2007) Haematologica 92, 795-802). Whereas some deletions were observed in several myeloma patients, other HDs were apparently patient-specific (Largo et al. (2007) Haematologica 92, 795-802). This pattern, which recurs in other cancers as well, suggests that some HDs are under positive selection in an evolving tumor, whereas other HDs of the same cancer, in the same patient, may be quasineutral, randomly retained deletions.

A salient property of an HD that involves DNA sequences not present elsewhere in the genome is that the HD cannot revert. HDs have a zero reversion frequency and do not change during tumor progression or therapy. This feature is of great consequence because it is the heterogeneity and constant transformation of cancer cells that causes the failures of many cancer therapies.

A major obstacle to drug-based therapies of human diseases that are both efficacious and substantially free of side effects is the massive interconnectedness and redundancy of molecular circuits in living cells. In the case of cancer, the problem is exacerbated by genomic instability of many, possibly most, cancers. This property increases heterogeneity of malignant cells in the course of tumor progression or anticancer treatment and is one reason for the failure of most drug-based cancer therapies (Weinberg, R A. (2006) The Biology of Cancer (Garland, New York); Vogelstein et al. (2004) Nat Med 10, 789-799). A few relatively rare cancers, such as testicular carcinoma, Wilm's kidney tumor, and some leukemias in children, can often be cured through chemotherapy but require cytotoxic treatments of a kind that cause severe side effects and are themselves carcinogenic (Einhom, L H. (2002) Proc Natl Acad Sci USA 99, 4592-4595; Hardman et al. (2001) The Pharmacological Basis of Therapeutics (McGraw-Hill, New York). Chemotherapy essentially uses cytotoxic drugs to kill fast-dividing cells. A disadvantage of chemotherapy is that the cancer cells are not specifically targeted and so non-cancer cells are also killed. This highly toxic treatment causes side effects that include pain, nausea and vomiting, diarrhea, malnutrition, and immune system depression. The negative effects highly constrain the amount that chemotherapy can be administered to a single patient.

Another cancer therapy is radiation therapy. This treatment employs the use of ionizing radiation to kill malignant cells. However, unlike other therapies, radiation is targeted to a certain region of the body and does not treat cancer cells throughout the body. Additionally, side effects include severe damage to epithelial surfaces, swelling, and fibrosis.

Monoclonal antibody therapy is another cancer treatment. This treatment uses monoclonal antibodies to specifically target cells using receptors found on the cell surface. Antibodies attach to these receptors to activate the immune system to attack malignant cancer cells. Prevention of tumor growth is also achieved by blocking specific cell receptors that are associated with unregulated growth or division of the tumor cell. However, this treatment requires a great deal of specificity in identifying receptors which is problematic due to the heterogeneity and volatility of cancer cells.

Several recent advances, including the use of antiangiogenic compounds and inhibitors of specific kinases, hold the promise of efficacious, curative therapies (Folkman, J. (2007) Nat Rev Drug Discov 6, 273-286; O'Hare et al. (2006) Curr Opin Genet Dev 16, 92-99; Sawyers, C. (2004) Nature 432, 294-297). Nevertheless, major human cancers are still incurable once they have metastasized.

Given the immutability of HDs and their high prevalence in cancer cells, a therapy capable of targeting cells with one or a multiplicity of HDs would provide significant advances in the art.

### SUMMARY OF THE INVENTION

In some embodiments, the present teachings provide methods, structures, vectors and compositions for activating a payload in cells having one or more homozygous DNA deletions (HDs). In some embodiments, methods of treating cancer by specifically delivering a payload, such as a toxin, to cancer cells comprising one or more particular HDs are provided.

In some embodiments, a method for selectively expressing a payload protein in a target cell having a homozygous DNA deletion (HD) is provided. The method comprises administering, to a population of cells comprising the target cell, a nucleic acid vector encoding at least two complementary protein fusion molecules and a payload protein such that the payload protein is selectively expressed in the target cell having the HD.

In some embodiments, the target cell can be a cancer cell. In some embodiments, the payload protein can be a toxic protein. In some embodiments, the payload protein is not expressed in cells lacking the HD.

In some embodiments, the target cell has two or more HDs, and the nucleic acid vector encodes at least four protein fusion molecules for targeting a set of the HDs and a payload protein such that the payload protein is selectively expressed in the target cell, and the payload protein is not expressed in cells lacking one or more HDs of the set.

In some embodiments, the nucleic acid vector can include a first nucleic acid sequence encoding a first protein fusion molecule including a first DNA binding domain and a first portion of a site-specific endonuclease; and a second nucleic acid sequence encoding a second protein fusion molecule including a second DNA binding domain and a second portion of the site-specific endonuclease, wherein the first and second DNA binding domains recognize adjacent DNA sequences that are not present in the target cell, and wherein upon binding of the first and second DNA binding domains to the adjacent DNA sequences, the first and second protein fusion molecules form and release an active site-specific endonuclease. In some embodiments, the nucleic acid vector can further include a third nucleic acid sequence encoding the payload protein, wherein the third nucleic acid is operably linked to an inducible promoter; and at least one recognition site for the site-specific endonuclease.

In some embodiments, the method can further include: expressing the first and second protein fusion molecules; reconstituting activity of the site-specific endonuclease in non-target cells; and administering to the population of cells an agent that induces expression of the payload protein in target cells.

In some embodiments, a nucleic acid vector for selectively expressing a payload protein in a target cell having an HD is provided. The nucleic acid vector comprises: a first nucleic acid sequence encoding a first protein fusion molecule comprising a first DNA binding domain and a first portion of a site-specific endonuclease; a second nucleic acid sequence encoding a second protein fusion molecule comprising a second DNA binding domain and a second portion of said site-specific endonuclease; a third nucleic acid sequence encoding a payload protein; and at least one recognition site for the site-specific endonuclease.

In some embodiments, upon binding of the first and second DNA binding domains to adjacent DNA sequences, the first and second protein fusion molecules interact to form an active site-specific endonuclease. In some embodiments, the adjacent DNA sequences comprise sequences that are deleted by the HD. In some embodiments, the adjacent DNA sequences are spaced apart such that their binding surfaces are on the same side of the DNA helix. In some embodiments, the first DNA binding domain and the second DNA binding domain can each be a zinc finger.

In some embodiments, the site-specific endonuclease can be a restriction endonuclease or a zinc finger nuclease.

In some embodiments, the vector can include multiple recognition sites for the site-specific endonuclease.

In some embodiments, the first protein fusion molecule can further include a first releasing domain, and the second protein fusion molecule can further include a second releasing domain. In some embodiments, the first releasing domain can include a C-terminal portion of ubiquitin (Ub) located between the first DNA binding domain and the first portion of a site-specific endonuclease, and the second releasing domain can include an N-terminal portion of the Ub located between the second DNA binding domain and the second portion of the site-specific endonuclease. In some embodiments, the first protein fusion molecule can further include a third releasing domain, and the second protein fusion molecule can further include a fourth releasing domain. In some embodiments, the third releasing domain can include an N-terminal portion of a ubiquitin-like protein (Ub1) located between the C-terminal portion of Ub and the first portion of a site-specific endonuclease, and the fourth releasing domain can include a C-terminal portion of the Ub1 located between the N-terminal portion of the Ub and the second portion of the site-specific endonuclease.

In some embodiments, the third nucleic acid sequence can be operably linked to an inducible promoter.

In some embodiments, the payload protein can be a toxic protein. In some embodiments, the toxic protein can be selected from the group consisting of a bacterial toxin and a plant toxin. In some embodiments, the toxic protein can be herpes simplex virus thymidine kinase (HSV-tk). In some embodiments, the payload protein can be a small compound dimerizer.

In some embodiments, the nucleic acid vector can be a DNA vector.

In some embodiments, the target cell can be a cancer cell.

In some embodiments a method of treating cancer in a patient is provided.

The method comprises: administering to the patient a nucleic acid vector encoding two complementary protein fusion molecules and a payload protein, wherein the payload protein is selectively activated in cancer cells having a homozygous DNA deletion (HD).

In some embodiments, the nucleic acid vector is inactivated in cells that do not have the HD.

In some embodiments, the payload protein induces terminal differentiation of the cancer cells. In some embodiments, the payload protein kills the cancer cells.

In some embodiments, the method further includes identifying the HD in cancer cells of a patient.

In some embodiments, the nucleic acid vector comprises: a first nucleic acid sequence encoding a first protein fusion molecule comprising a first DNA binding domain specific and a first portion of a site-specific endonuclease; a second nucleic acid sequence encoding a second protein fusion molecule comprising a second DNA binding domain and a second portion of the site-specific endonuclease, wherein the first and second DNA binding domains recognize adjacent DNA sequences, wherein the adjacent DNA sequences are deleted by the HD, and whereupon binding of the first and second DNA binding domains to the adjacent DNA sequences, the first and second protein fusion molecules can interact to form an active site-specific endonuclease; a third nucleic acid sequence encoding a payload protein operably linked to an inducible promoter; and at least one recognition site for the site-specific endonuclease.

In some embodiments, the method further includes expressing the first and second protein fusion molecules in non-cancer cells of the patient. In some embodiments, the method further includes reconstituting activity of the site-specific endonuclease in non-cancer cells of the patient. In some embodiments, the method further includes verifying the status of the nucleic acid vector in a sample of cells from the patient. In some embodiments, the method further includes administering to the patient an agent that induces expression of the payload protein in cancer cells of the patient.

### BRIEF DESCRIPTION OF THE DRAWINGS

FIGS. 1A-C generally depict one embodiment of deletion-specific targeting (DST). (A) Chromosome pairs 1 and 2 (arbitrarily numbered) in a diploid cell. (B) The same chromosomes as in (A) in a cell that contains two homozygous deletions (HDs), termed HD1 and HD2. The nonoverlapping parts of hemizygous deletions are shown as a crosshatched pattern (xxx). Their overlapping parts, which comprise, respectively, HD1 and HD2, are shown as a diagonal line pattern on chromosome 1 (\\\\) and chromosome 2 (////). (C) Outline of an embodiment of DST.

FIGS. 2A-H generally depict one embodiment of DST devices and their implementation. (A) DST-fusion-1 (DST-f1). (B) DST-fusion-2 (DST-f2). (C) The ORFs of one embodiment of a DST vector containing five ORFs. The ORF encoding a “payload” is depicted as a rectangle labeled “P.” P₁, P₂, P₃, P₄ and P₅ denote promoters. (D) DST vector enters normal (non-target) cells, which contain either one or both of the DNA segments DNA-1 and DNA-2. (E) Binding of DST-f1 and DST-f2 to DNA-1 and reconstitution of restrictase-1 (r1). DUB, deubiquitylating enzyme; UBLP, Ub1-specific protease. (F) Conditional destruction of the DST vector. (G) Binding of DST-f3 and DST-f4 to DNA-2 and reconstitution of restrictase-2 (r2). (H) Events in a target cell, i.e., one that contains both HD1 and HD2, that has received a DST vector.

FIGS. 3A-B generally depict an overview of one embodiment of DST. (A) Flow chart depicting one embodiment of DST in normal (non-target) cells. (B) Flowchart depicting one embodiment of DST in target cells.

### DETAILED DESCRIPTION OF SOME EMBODIMENTS

Various embodiments disclosed herein are generally directed towards targeting homozygous DNA deletions (HDs) in cells and related methods, structures, vectors and compositions. A new approach, termed deletion-specific targeting (DST), has been developed that employs HDs (not their effects on RNA/protein circuits, but deletions themselves) as the targets of payload activity. DST employs an HD as a “negative” target of cells (Varshavsky (2007) Proc. Natl. Acad. Sci. USA 104(38):14935-14940, which is incorporated herein by reference in its entirety).

The DST strategy is implemented through a molecular “DST circuit” that results in the activation of one or more payloads of a “DST vector” only in cells having one or more HD(s). The payload can be any anything that can be encoded or carried in a vector, and that is desired to be delivered or expressed in a target cell. Typically, the payload can be a protein for which expression is desired in cells having the targeted HD(s). A Boolean output of a DST circuit is used to either inactivate a DST vector, thereby precluding activation of its payload, or to activate the payload if the circuit reports the presence of one or more HDs in a cell. Thus, the payload is not activated in cells lacking the targeted HD(s) and, in fact, is actively inactivated. In some embodiments, the activated payload can cause, directly or indirectly, terminal differentiation or killing of cells containing the targeted HD(s). In other embodiments, the payload can be a biomarker, such as, for example, a fluorescent protein such as green fluorescent protein (GFP). In some embodiments, the activated payload can label cells having the targeted HD(s). In some embodiments, the activated payload can, for example, be an enzyme involved in a biochemical pathway.

In some embodiments, methods for targeting one or more HDs in cells are disclosed. The methods generally involve a nucleic acid vector (the “DST vector”) encoding at least two complementary multidomain protein fusions, as well as a “payload” protein. Each fusion includes a DNA binding domain which recognizes a given sequence of DNA (“recognition sequence”) that is absent (deleted) in a target cell as a result of an HD. Complementary fusions have DNA binding domains which recognize adjacent DNA sequences in wild type cells. Preferably, each recognition sequence is at least about 9 bp long. The fusion also includes “releasing” domains that are linked to the DNA binding domain. For example, the releasing domains can comprise split-ubiquitin-based domains. Split ubiquitin proteins are described in, for example, U.S. Pat. Nos. 5,503,977 and 5,585,245, which are herein incorporated by reference in their entireties. The fusion further includes a “split” site-specific endonuclease-based domain. The sequence recognized and cleaved by this endonuclease is present in multiple copies in the DST vector, and is absent from target cell DNA, such as mammalian DNA in embodiments targeting HDs in mammalian cells.

In some embodiments, the DST vector is introduced into a population of cells. Once inside a cell, the DST vector expresses the complementary protein fusions, but does not express, as yet, the vector payload. The fusions, through their DNA binding domains, recognize their corresponding recognition sequences that are present in normal cells but are absent (deleted) in a cell with an HD that is targeted, such as a cancer cell with one or more specific HDs. The binding of the complementary protein fusions to two adjacent DNA sequences in a cell lacking the targeted HD brings together the otherwise “split” halves of the other components of the complementary fusions, including the split site-specific endonuclease. The act of bringing together complementary fusions, i.e., two halves of a split site-specific endonuclease, reconstitutes the endonuclease activity, releases the reconstituted endonuclease, and results in a feedback circuit in which the site-specific endonuclease destroys, through multiple cuts, the DST vector that encodes the fusions and the open reading frame encoding the vector's payload. As a result, if the DST circuit senses the presence of DNA sequences that are known to be deleted in a target cell because of a targeted HD (for example, in a cancer cell and not in normal cells), the DST vector will be destroyed. Thus, the payload protein is not expressed in cells lacking the targeted HD. If the DST circuit does not sense the presence of the DNA sequences, the complementary fusions are not brought together, endonuclease activity is not reconstituted, and the DST vector remains intact and the payload protein is subsequently expressed. Thus, the DST vector remains intact and the payload is expressed only in cells lacking the given DNA segments, i.e., cells having the targeted HD.

In some embodiments, methods and compositions are provided for activating a payload in cells having one or more HDs. For example, a delayed activation of the DST vector's payload can be carried out once the DST vector has been eliminated from cells that do not contain the DNA recognition sequences of the DST vector fusions i.e., cells having the targeted HD(s). Activation of the payload generally results in expression of a desired protein (the “payload protein”) in the target cell. In some embodiments, activation of the payload results in terminal differentiation or death of the cell containing the activated payload. In some embodiments, once a payload protein is expressed, payload activity can be intrinsic, i.e., occurring naturally. In other embodiments, payload activity can be stimulated, such as, for example, by administering an agent.

As will be appreciated by one of skill in the art, the ability to specifically target and activate a vector payload in a cell having an HD can have great benefit, especially for treating a disease involving undesirable cells harboring an HD, such as cancer cells. For example, the methods and compositions disclosed herein are beneficial for, inter alia, causing terminal differentiation or death of cells having an HD. In some embodiments, methods and compositions disclosed herein are beneficial for providing, for example without limitation, killing cancer cells while leaving healthy cells untouched. In some embodiments, the methods and compositions disclosed herein can be used to identify cells containing at least one HD, and more generally to identify the absence of particular DNA sequences in a sample.

In some embodiments, the methods and compositions are also applicable to the treatment of cancer. For example, a patient diagnosed with cancer can be selected for treatment with DST. One or more homozygous DNA deletions are identified in cancer cells of the patient. In this example, two HDs, arbitrarily named “HD1” and “HD2” are identified in cancer cells of the patient, and corresponding sequence segments “DNA-1” and “DNA-2” are identified as DNA segments deleted by HD1 and HD2, respectively (DNA-1 represents a segment of DNA deleted by HD1, and DNA-2 represents a segment of DNA deleted by HD2). Zinc finger (ZF) domains which recognize adjacent sequences in the DNA segments deleted by the HDs can be identified. For example, for targeting a cell having HD1 and HD2, zinc finger domains “ZF1-1,” “ZF1-2,” “ZF2-1” and “ZF2-2,” which recognize adjacent sequences in DNA-1 and DNA-2, can be used. In some embodiments, ZF1-1 and ZF1-2 recognize adjacent sequences in DNA-1, and ZF2-1 and ZF2-2 recognize adjacent sequences in DNA-2.

A DST vector for targeting the one or more HDs, for example, HD1 and HD2, is provided. In some embodiments, the DST vector can include ORFs for protein fusion molecules that bind to adjacent sequences in deleted DNA segments and reconstitute site-specific endonuclease activity. For example, a DST vector can include ORFs for four fusions: two complementary fusions that recognize DNA-1 (DST-f1 and DST-f2), and two complementary fusions that recognize DNA-2 (DST-f3 and DST-f4). The protein fusions DST-f1, DST-f2, DST-f3 and DST-f4 include the ZF domains ZF1-1, ZF1-2, ZF2-1 and ZF2-2, respectively. Each fusion also includes a “split site-specific endonuclease” domain. The binding of DST-f1 and DST-f2 to their recognition sequences in cells lacking HD1 brings together two halves of a split site-specific endonuclease and reconstitutes the activity of the site-specific endonuclease “r1.” Similarly, the binding of DST-f3 and DST-f4 to their recognition sequences in cells lacking HD2 brings together two halves of a split site-specific endonuclease and reconstitutes the activity of the site-specific endonuclease “r2.” The DST vector includes multiple cleavage sites for r1 and r2. The cleavage sites for r1 and r2 can be the same or different, and are not present in the human genome. Each of the fusions also includes two “releasing” domains between the ZF domain and split site-specific endonuclease domain. The releasing domains facilitate release of the reconstituted r1 and r2. The releasing domains can include, for example, a split Ub domain and a split Ub1 domain. The binding of DST-f1 and DST-f2 to their recognition sequences in cells lacking HD1 brings together two halves of the split Ub and Ub1 and reconstitutes the activities of the Ub and Ub1. Similarly, the binding of DST-f3 and DST-f4 to their recognition sequences in cells lacking HD2 brings together two halves of the split Ub and Ub1 and reconstitutes the activities of the Ub and Ub1. The DST vector also includes an ORF for a payload protein. For treatment of cancer, the payload protein can be, for example without limitation, a cytoxic protein or a conditionally cytotoxic protein. In various embodiments, expression of the payload is controlled by a non-leaky inducible promoter.

In various embodiments, a patient diagnosed with cancer having homozygous DNA deletions HD1 and HD2 is given a therapeutically effective intravenous dose of a DST vector for targeting HD1 and HD2. In some embodiments, the DST vector payload is a cytotoxic protein such as, for example, diphtheria toxin. The fusions are expressed in the cells of the patient, and site-specific endonuclease activity is reconstituted in cells lacking HD1 and/or HD2 by the binding of the fusions to their recognition sequences. A suitable time period is allowed for destruction of the DST vector in cells lacking one or both HDs. In various embodiments, a sample from the patient can be analyzed to verify the status of the DST vector. After destruction of the DST vector in non-cancer cells has been verified, an agent for inducing expression of the payload protein can be administered to the patient. The cytotoxic protein is expressed and/or activated in cancer cells only, killing the cancer cells. The DST circuit described above can be applied to delivery of payloads to target cells with one or more HDs in context other than cancer, as will be appreciated by the skilled artisan.

The logic of DST makes possible an incremental and essentially unlimited increase in the selectivity of therapy by, for example, increasing the number of HDs targeted. Implemented in a clinical setting, DST may be curative and substantially free of side effects, as it can be used to target only disease associated cells, although it need not be in all circumstances. The DST devices disclosed herein target the HD itself. The DST circuit is modular, and the circuit's selectivity may be increased by adding components to target additional HDs. A major advantage of DST is its essentially unlimited selectivity. The number of known cancer-associated HDs is already large, and specific cancers in individual patients can be relied on to contain at least one and typically at least two homozygous deletions that satisfy the parameters of DST. The DST strategy is independent of considerations that underlie other therapeutic approaches. For example, DST does not involve a function of deleted DNA, or its levels of expression in normal cells, or tumorigenic alterations of RNA/protein circuits in cancer cells, or cell-surface differences between them and normal cells.

The above and additional embodiments are discussed in more detail below, after a brief discussion of some of the terms used in the specification.

### DEFINITIONS

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques of described herein are those well known and commonly used in the art.

As utilized in accordance with the embodiments provided herein, the following terms, unless otherwise indicated, shall be understood to have the following meanings:

As used herein, the terms “homozygous DNA deletion,” “homozygous deletion” and “HD” are used interchangeably and refer to a deletion of one or more base pairs in a double-stranded DNA. Preferably, an HD is at least about 100 bp.

As used herein, the term “deletion-specific targeting vector” and “DST vector” are used interchangeably and refer to a nucleic acid vector that encodes the components of the DST circuit. In some embodiments, a DST vector encodes at least two complementary protein fusion molecules and a payload protein. A DST vector including an ORF for a payload protein includes multiple recognition sites for a site-specific endonuclease whose activity is reconstituted in the absence of a targeted HD.

As used herein, the term “target cell” refers to a cell having each and every one of the HDs targeted by a particular DST circuit.

As used herein, the term “payload” refers to a molecule that can be encoded or carried in a vector, and that is desired to be delivered or expressed in a target cell. Typically, the payload is a protein for which expression is desired in a target cell. In some embodiments, the payload and fusions are encoded by the same DST vector. In other embodiments, the payload and fusions can be encoded by different vectors.

As used herein, the term “adjacent” means near or close to but not necessarily touching. In some embodiments, adjacent DNA sequences can be spaced apart so long as the desired biological activity is maintained. For example, in the context of complementary fusions, recognition sequences can be spaced apart such that the fusions can interact and reconstitute a desired activity, for example, endonuclease activity. In other embodiments, adjacent DNA sequences can be touching.

As used herein, the term “cancer” is intended to mean a class of diseases characterized by the uncontrolled growth of aberrant cells, including all known cancers, and neoplastic conditions, whether characterized as malignant, benign, soft tissue or solid tumor. Specific cancers include digestive and gastrointestinal cancers, such as anal cancer, bile duct cancer, gastrointestinal carcinoid tumor, colon cancer, esophageal cancer, gallbladder cancer, liver cancer, pancreatic cancer, rectal cancer, appendix cancer, small intestine cancer and stomach (gastric) cancer; breast cancer; ovarian cancer; lung cancer; renal cancer; central nervous system (CNS) cancer, including brain cancer; prostate cancer; hematopoietic neoplasms such as leukemia, lymphoma and melanoma; skin cancers, eye cancers, and the like.

As used herein, “pharmaceutically or therapeutically acceptable carrier” refers to a carrier medium which does not interfere with the effectiveness of the biological activity of the active ingredients and which is minimally toxic to the host or patient.

As used herein, “therapeutically- or pharmaceutically-effective amount” as applied to the disclosed compositions refers to the amount of composition sufficient to induce a desired biological result. That result can be alleviation of the signs, symptoms, or causes of a disease, or any other desired alteration of a biological system. For example, the result can involve a decrease and/or reversal of cancerous cell growth.

The DST Circuit and its Operation.

The DST strategy involves a nucleic acid vector (the DST vector) encoding at least two multidomain protein fusions, as well as a “payload” protein. Briefly, in some embodiments, the fusions are designed to bind to DNA sequences within a segment of DNA that is absent (deleted) in target cells due to a given HD. Upon DNA binding, complementary protein fusions are brought into close enough proximity that they are able to reconstitute an enzymatic activity which destroys the DST vector, typically a site-specific endonuclease, which subsequently cleaves and destroys the DST vector encoding the fusions and payload. If the segment of DNA recognized by the fusions is not present, such as in target cells having the given HD, the DST vector remains intact, and the payload can be activated in these target cells.

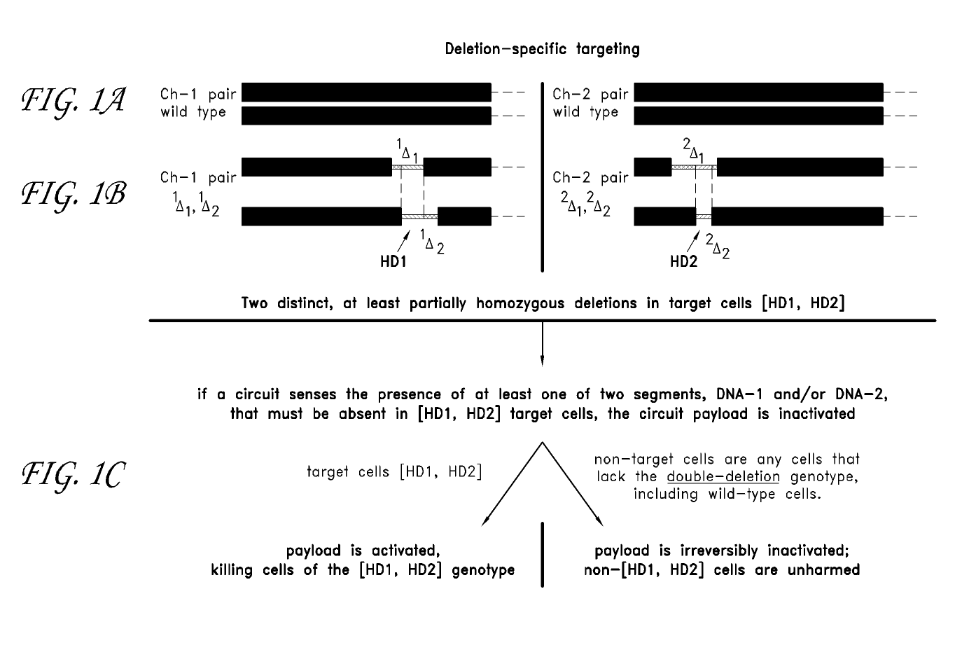

One embodiment of a DST circuit is schematically depicted in FIGS. 1-3. While the embodiment shown in FIGS. 1-3 involves targeting of a double-deletion phenotype (two HDs in a cell), the skilled artisan will readily appreciate that the DST strategy can be employed to target single-, triple-, quadruple-deletion, and other multiple-deletion phenotypes in a similar manner. In some embodiments, a DST circuit can target at least one HD in target cells. In other embodiments, a DST circuit can target at least two, three or four distinct HDs in target cells.

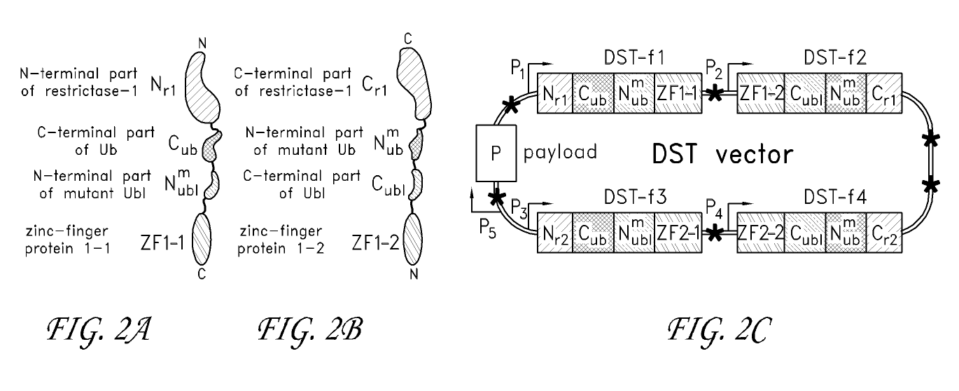

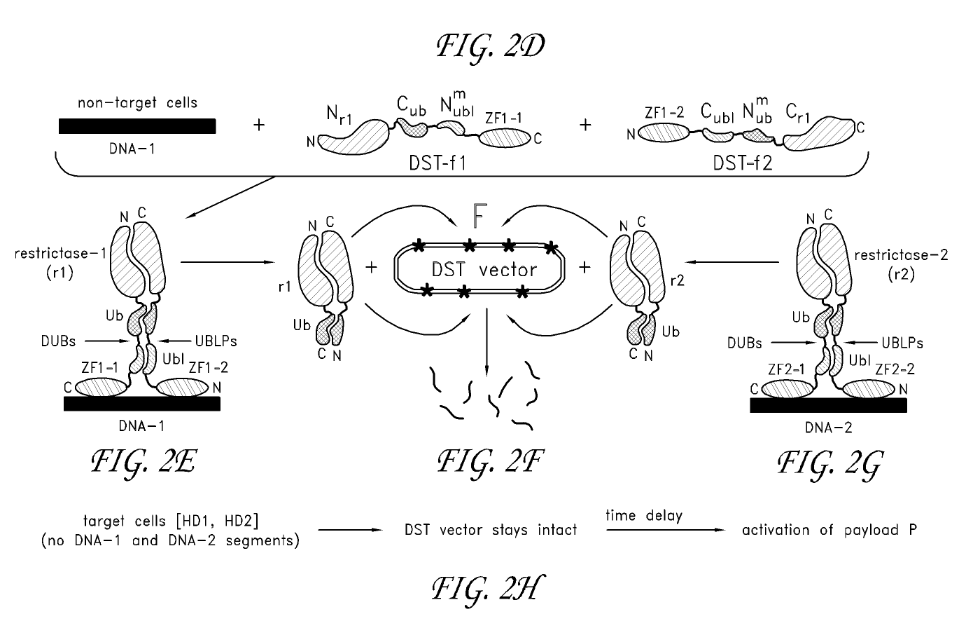

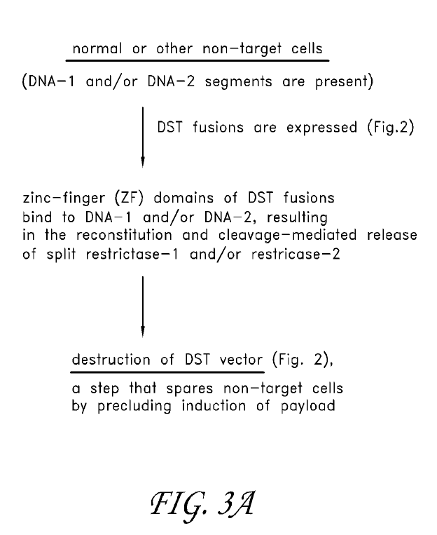

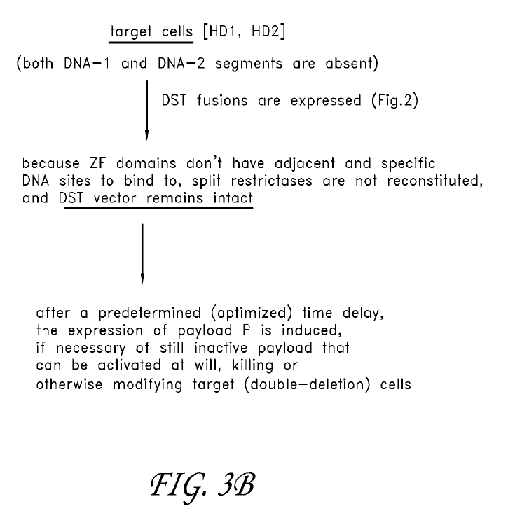

For the embodiment depicted in FIGS. 1-3, two homozygous deletions are identified, termed HD1 and HD2, in a population of target cells (FIG. 1). These deletions removed DNA segments that are termed DNA-1 and DNA-2 (FIGS. 2 and 3). HD1 and HD2 preferably encompass unique DNA sequences and their minimally acceptable sizes can be as small as about 100 bp. In other embodiments, one, two, three, four or more HDs can be identified for targeting using the DST strategy described herein.

FIG. 1B depicts a pair of overlapping deletions, ¹Δ₁ and ¹Δ₂ (the wild-type chromosome pairs are shown in FIG. 1A). The two HDs result from overlapping hemizygous deletions ¹Δ₁ and ¹Δ₂ in chromosome 1 and ²Δ₁ and ²Δ₂ in chromosome 2. A segment of deleted DNA in common between ¹Δ₁ and ¹Δ₂ is termed DNA-1, and the corresponding homozygous deletion is termed HD1. ²Δ₁ and ²Δ₂ are another pair of hemizygous deletions. In FIG. 1B, they are located on a different pair of chromosomes from ¹Δ₁ and ¹Δ₂, but they can also be located on the same pair of chromosomes as the first set of hemizygous deletions. DNA-2 is the segment of deleted DNA in common between ²Δ₁ and ²Δ₂, and the corresponding (second) homozygous deletion is termed HD2 (FIG. 1B). The nucleotide sequences and lengths of DNA-1 and DNA-2 are not meant to be restricted to any particular sequence or length, and will vary depending on the HDs to be targeted.

DNA-1 and DNA-2 are absent in [HD1, HD2] target cells (FIGS. 1A-1C). If the DST circuit senses the presence of at least one of the two segments, DNA-1 and/or DNA-2, that are necessarily absent in the [HD1, HD2] target cells, the DST circuit payload is irreversibly inactivated. As a result, the payload in non-[HD1, HD2] cells, i.e., cells that lack the double-deletion genotype, is not activated, and the cells are not affected (FIG. 1C, right panel). In the [HD1, HD2] target cells, the payload can be activated, killing cells of the [HD1, HD2] genotype (FIG. 1C, left panel). Thus, in the depicted embodiment, both HDs must be present in a cell for the payload to be activated.

In other embodiments, the DST circuit is designed to target a single HD, and the DST circuit payload is irreversibly inactivated if the HD is not present. In addition, in other embodiments, the payload may only be activated if additional HDs are present in a target cell. As in DST circuits designed to target multiple HDs, if the DST circuit senses the presence of at least one of the segments of DNA that are necessarily absent from the target cells, i.e., cells having the desired HD genotype, the DST circuit payload is irreversibly inactivated. Thus, the DST circuit payload is only activated in cells having a desired HD genotype.

FIGS. 2 and 3 depict one embodiment of DST constructs for targeting HD1 and HD2, and the circuit they comprise. In FIGS. 2 and 3, the segments of DNA that had been removed from wild-type cells as a result of homozygous deletions HD1 and HD2 (FIG. 1) are called, respectively, DNA-1 and DNA-2.

In the depicted embodiment, one “half” of a DST circuit is implemented by two “complementary” protein fusions termed DST-f1 and DST-f2 (FIGS. 2A and 2B). The other, mechanistically identical “half” of the circuit is implemented by fusions termed DST-f3 and DST-f4 (FIG. 2G). In general, two complementary protein fusions target one distinct HD. Therefore, in embodiments where only one HD is targeted, the whole circuit is implemented by two “complementary” protein fusions, and additional fusions are not required. Where more two or more HDs are targeted, four or more protein fusions can be used for the DST circuit.

According to various embodiments, each protein fusion includes, beginning at its N terminus, a split site-specific endonuclease domain, releasing (e.g., split-ubiquitin-based) domains, and a DNA-binding domain. Each of these domains is described in more detail below using the fusion DST-f1 depicted in FIG. 2A as an example.

In some embodiments, each fusion molecule includes four domains. For example, beginning at its N terminus, the first domain of DST-f1 shown in FIG. 2A comprises an N-terminal fragment of a site-specific endonuclease (restrictase-1, or r1) whose specific DNA cleavage site is absent from human the genome of non-target cells but is present, at multiple locations, in the DNA of the DST vector (FIG. 2C). The specific DNA cleavage sites in the DST vector are shown as asterisks in the vector shown in FIG. 2C. While in this example an endonuclease is used, other enzymatic activities that specifically destroy the DST vector can be used. The fragment of the site-specific endonuclease is referred to as a “split site-specific endonuclease” or “split restrictase.” Similarly to the previously characterized “helper-dependent” split proteins (Johnsson and Varshavsky (1994) Proc Natl Acad Sci USA 91:10340-10344, which is incorporated herein by reference in its entirety), the split restrictase r1 is constructed in such a way that a moderate-level coexpression of its N-terminal and C-terminal fragments (FIGS. 2A and B) cannot reconstitute the enzymatically active r1 restrictase, but it can be reconstituted if these fragments are brought into spatial proximity, as shown in FIG. 2E. Thus, importantly, the split site-specific endonuclease does not spontaneously reconstitute unless the two complementary portions are brought into spatial proximity. A number of suitable site-specific endonucleases are known in the art and can be used. One example, without limitation, of a site-specific endonuclease with requisite cleavage specificity is yeast SceI, which cuts DNA at an 18-bp-long recognition site (Jasin, M. (1996) Trends Genet 12, 224-228). SceI is a member of the large and extensively characterized class of “homing” endonucleases, which mediate, in particular, the activity of selfish genetic elements (reviewed in Stoddard, BL. (2006) Quart Rev Biophys 38, 49-95). Another class of site-specific endonucleases that can also be used to design a split restrictase includes artificial (engineered) ZF nucleases (ZFNs) (Porteus et al. (2003) Science 300, 763; Porteus et al. (2006) Nat Biotech 23, 967-973; Umov et al. (2005) Nature 435, 646-651; Szepek et al. (2007) Nat Biotechnol 25, 786-793; Miller et al. (2007) Nat Biotech 25, 778-785; Johnsson (1994) Proc Natl Acad Sci USA 91, 10340-10344; Michnick et al. (2007) Nat Rev Drug Discov 6, 569-582; Zhang (2004) Cell 119, 137-144; Magliery (2005) J Am Chem Soc 127, 146-157; Kerppola, T K. (2006) Nat Rev Mol Cell Biol 7, 449-556; Galameau et al. (2002) Nat Biotechnol 20, 619-622; Ooi et al. (2006) Biochemistry 45, 3620-3625 and references therein). In other embodiments, reconstituted nucleases, which mediate the destruction of the DST vector in non-target cells (FIG. 2), can comprise, for example, split recombinases such as Cre of the phage P1. In embodiments using Cre, a corresponding Cre-sensitive DST vector would contain multiple copies of loxP, the target of Cre, or (for example) a silent, loxP-containing, Cre-activated transcriptional promoter upstream of a gene encoding an intact (nonsplit) homing endonuclease. Thus, reconstituted Cre would activate transcription of the homing endonuclease.

The next domain of DST-f1 shown in FIG. 2A is a releasing domain. In DST-f1, one releasing domain comprises Cub, a C-terminal half of the 76-residue Ub moiety. This Ub moiety is referred to as a “split-Ub,” and has previously been described a part of the previously characterized split-Ub sensor (Johnsson et al. (1994) Proc Natl Acad Sci USA 91, 10340-10344).

The third domain of DST-f1 shown in FIG. 2A is another releasing domain. In DST-f1, the second releasing domain comprises a mutated N-terminal half of a Ub-like (Ub1) protein such as, for example without limitation, NEDD8 or SUMO (Kerscher (2006) Annu Rev Cell Dev Biol 22, 159-180), that is a part of the additional (Ub1-based) split-protein sensor in the current DST design. Linking split-Ub and split-Ub1 in the same pair of fusions (FIGS. 2A and B) makes possible a conditional cleavage of two polypeptide chains at once (after the last residue of Ub in DST-f1 and after the last residue of Ub1 in DST-f2) and the resulting release of (reconstituted) restrictase r1, as shown in FIGS. 2E and F. A properly placed and double (as distinguished from single) proteolytic cleavage ensures release of the reconstituted (previously split) restrictase r1 moiety (FIG. 2E). A single cleavage would not release the reconstituted (previously split) restrictase r1 moiety, because the Ub halves and the Ub1 halves of DST-f1/DST-f2 remain associated at this stage. Because all Ub-like proteins share the central feature of the Ub fold (a short α-helix over a β-fold), and because Ub fold-destabilizing mutations (in the N-terminal half of Ub) were previously characterized with the split-Ub sensor (Johnsson et al. (1994) Proc Natl Acad Sci USA 91, 10340-10344), a split Ub1 protein has the required properties and is used.

Those of skill in the art will appreciate that the location of the above Ub and Ub1 moieties are interchangeable in the fusion.

In some embodiments, a split Ub1 moiety (instead of a second split-Ub moiety) can be used in DST-f1/DST-f2 fusions (FIGS. 2A and B) to bypass the possible problem of intramolecular (as distinguished from intermolecular) reconstitution of the Ub moiety. In other words, if the Cub half of Ub is followed, in DST-f1, by the N_{ub} half of Ub, the two halves may be able to associate intramolecularly, an event to avoid: hence the use of split Ub1, which would be designed to be incapable of cross-associating with split Ub. However, if the linker sequences (sequences between the C_{ub} half of Ub and N_{ub} half of Ub) involved are made sufficiently short, for example, about 2 to about 3 amino acid residues in length, steric constraints can prevent intramolecular folding and the (undesirable) reconstitution of the Ub moiety. Thus, in other embodiments, DST-f1 and/or DST-f2 fusions comprising two otherwise identical split-Ub moieties oriented in opposite directions, instead of split-Ub and split-Ub1, can be used.

The fourth and last domain of DST-f1 shown in FIG. 2A comprises ZF1-1, a DNA binding domain. In the depicted embodiment, ZF1-1 is a zinc finger (ZF) protein domain that recognizes specific DNA sequences (see, e.g., Choo et al (1994) Nature 372, 642-645; Jamieson, A C, Miller, C J & Pabo, C O. (2003) Nat Rev Drug Discov 2, 361-368; Blancafort et al. (2005) Proc Natl Acad Sci USA 102, 11716-11721; Papworth et al. (2006) Gene 366, 27-38. ZF domains are well known in the art, and can be selected for binding to a given DNA sequence. The ZF domains ZF1-1 and ZF1-2, of the fusions DST-f1 and DST-f2, respectively (FIGS. 2A and B), are designed to bind to two adjacent sequences of DNA-1 (FIG. 2D). See, for example, Stains et al., (2005) J Am Chem Soc 127, 10782-10783; Ghosh et al., (2006) Mol BioSyst 2, 551-560. Preferably, the two ZF-binding sequences of DNA-1 are each preferably about 9 bp, but could be from about 9 bp to about 12 bp or greater. In some embodiments, the sequences are greater than about 9 bp. The two ZF-binding sequences of DNA-1 are spaced apart to position their binding surfaces on the same side of the DNA double helix. In some embodiments, the junction between a ZF domain and a split-Ub (or a split Ub-like) module in a DST fusion can be designed to activate a ZF-linked cryptic degron upon the fusion's cleavage, resulting in a short-lived ZF and multiple cycles of split-nuclease reconstitution on a single-copy DNA segment.

FIG. 2B depicts DST-f2, a fusion “complementary” to DST-f1. The two fusions can interact as a result of specific binding of the fusions' ZF1-1 and ZF1-2 moieties to adjacent DNA sequences of DNA-1, as described above and in FIGS. 2E and F. However, in the absence of binding to DNA, the two fusions do not interact.

The DST strategy can be carried out in a number of formats. Common to such formats is the use of DNA-based expression constructs. DNA-based expression constructs are genetic elements which can replicate and express the desired proteins within the experimental system employed. The starting material for such expression constructs will typically be a well-characterized expression vector (e.g., a plasmid such as the one depicted in FIG. 2C) which contains regulatory elements (e.g., the origin of replication, promoters, etc.) which are suitable for use within a mammalian cell. Many expression vectors suitable for use in mammalian cells have been reported and are used routinely by those skilled in the art. A review of this fundamental information will not be undertaken here.

In some embodiments, a DST vector includes least three open reading frames (ORFs); two ORFs for the fusions and one ORF for the payload. In other embodiments, the ORFs for the fusions and payload can be on separate vectors. FIG. 2C depicts the ORFS of one embodiment of a DST vector. The depicted DST vector includes five ORFs: one ORF for each of the fusions (to detect DNA-1 and DNA-2, if present), and an ORF encoding the payload. In other embodiments, a DST circuit targeting a single HD can employ a DST vector including at least three ORFs. Other embodiments can include more ORFs depending on the number of targeted HDs and payloads. In other embodiments, the ORF for the payload can be on a separate vector from the ORFs for the fusions. In some embodiments, the ORFs for each fusion can be present on separate vectors. In some embodiments, the ORF for complementary fusions can be present on the same vector.

The ORF(s) encoding the “payload” (e.g., a conditionally toxic protein, a toxic protein, a biomarker, and enzyme, etc.) is depicted as a rectangle with a “P.” The other ORFs of the DST vector depicted in FIG. 2C encode DST-f1 (FIG. 2A), DST-f2 (FIG. 2B), DST-f3, and DST-f4 (FIG. 2G). The latter pair of ORFs is similar to the one encoding DST-f1 and DST-f2, but includes the following differences: ZF2-1 and ZF2-2 ZF domains of DST-f3 and DST-f4 recognize DNA sequences distinct from those recognized by ZF1-1 and ZF1-2 (FIGS. 2A and B). Preferably, the DNA sequences recognized by DST-f3 and DST-f4 are about 9-bp or greater. The split restrictase r2 of DST-f3/DST-f4, although of the same cleavage specificity as the restrictase r1, does not “cross-reconstitute” with it because of the different binding specificities. The asterisks shown on the DST vector denote the cleavage site recognized by r1 and r2 that is present in multiple copies in the DST vector's DNA but is absent from human DNA. Any number of cleavage sites (greater than one) can be present in the DST vector for inactivation of the vector in non-target cells. If the fusion ORFs and payload ORFs are located on different vectors, the cleavage site recognized by the restrictase is present in multiple copies on at least the vector comprising the ORF for the payload. In some embodiments, a cleavage site recognized by the restrictase is located between the ORF for the payload and the promoter regulating expression of the payload.

In some embodiments, the DST vector is delivered into cells. Preferably, the DST vector is delivered into cells as nonspecifically as possible, either as, for example, a part of DNA viruses, or by any other route (e.g., without limitation, a liposome-DNA complex, chemical transfection, lentivirus, etc.). Preferably, the route of administration involves a nonreplicating delivery vector to minimize immunogenicity of the procedure and maximizes its efficiency (Verma et al. (2005) Annu Rev Biochem 74, 711-738; McCormick, F. (2005) Oncogene 24, 7817-7819; Vasileva et al (2005) Nat Rev Microbiol 3, 837-847; Limberis et al. (2006) Proc Natl Acad Sci USA 103, 12993-12998; Schillinger et al. (2005) Proc Natl Acad Sci USA 102, 13789-13794). Methods for delivering DNA into cells are known in the art. Non-specific delivery of the DST vector is suitable for the methods disclosed herein.

Expression of a fusion molecule can be regulated by any suitable promoter known in the art for regulating transcription in mammalian cells. In FIG. 2C, the promoters P₁, P₂, P₃ and P₄ are the promoters for driving expression of the ORFs for DST-f1, DST-f2, DST-f3 and DST-f4, respectively. In some embodiments, the promoters controlling expression of the fusions can be constitutive promoters. In other embodiments, the promoters are inducible promoters. The payload P is preferably under control of an inducible promoter. In FIG. 2C, the inducible promoter P₅ controls expression of payload P. A variety of suitable constitutive and inducible promoters are known in the art, such as, for example without limitation, the P_{CMV} promoter, based on a strong transcriptional promoter of the human cytomegalovirus, or the P_{tetO} promoter, which can be regulated as a doxycycline/tetracycline-inducible promoter.

The general operation of one embodiment of a DST circuit in a normal (non-target) cell, which contains both DNA-1 and DNA-2, is generally depicted in FIGS. 2D-2F. In the depicted embodiment, the DST vector enters normal (non-target) cells, which contain either one or both of DNA segments, DNA-1 and DNA-2, that are absent in target cells owing to the homozygous deletions HD1 and HD2 (FIG. 2D). Only DNA-1 and its ligands DST-f1 and DST-f2 are shown in D. The other pair of DST fusions, DST-f3 and DST-f4, which recognize DNA-2, is shown in G. As shown in FIGS. 2D and 2E, upon expression by DST vector of DST-f1 and DST-f2, their DNA-recognizing domains ZF1-1 and ZF1-2 locate and bind to their adjacent recognition sequences on human DNA-1 (FIG. 2E). Coexpression of DST-f1 and DST-f2, and the binding of ZF1-1 and ZF1-2 to the above two sequences of DNA-1 bring the rest of DST-f1 and DST-f2 fusions into close proximity, thereby inducing reconstitution of the split restrictase r1, of Ub, and of Ub1, as shown in FIGS. 2D-F. See, for example, Stains et al. (2005) J Am Chem Soc 127, 10782-10783; Ghosh et al. (2006) Mol BioSyst 2, 551-560. This reassembly leads to cleavages by constitutively present deubiquitylating (DUB) enzymes after the last residue of Cub (Johnsson et al. (1994) Proc Natl Acad Sci USA 91, 10340-10344) and by Ub1-specific proteases (UB1Ps, also constitutively present) (Kerscher et al. (2006) Annu Rev Cell Dev Biol 22, 159-180) after the last residue of C_{ub1}, as shown in FIGS. 2E and F. Because of the way in which C_{ub} and C_{ub1} are placed in DST-f1/DST-f2, these cleavages liberate the active (reconstituted) restrictase r1 moiety from its association with human DNA and lead to r1-mediated cleavage of the DST vector (FIG. 2F). This vector encodes, in particular, the DST-f1 and DST-f2 fusions and, in addition, contains multiple cleavage sites, indicated by asterisks in FIG. 2C, for restrictases r1 and r2, whose cleavage specificities are the same. In other embodiments they cleavage specificities of r1 and r2 can be different. The released nucleases becomes free to target its cleavage sites in DST vector, digesting it to small fragments and thereby halting the expression of its ORFs, and preventing expression of vector's payload P (FIG. 2F). Note that the payload's expression had not been induced as yet at this stage. Thus, the overall result of r1 activation (FIGS. 2E and F) is the destruction of DST vector, and thus the prevention of induction of its payload P under conditions in which the above DST circuit had detected the presence of DNA-1.

In some embodiments, the DST circuit is designed to target a single HD. In embodiments where a single HD is targeted, only two complementary fusions (e.g., DST-f1 and DST-f2) are required. In other embodiments, two or more HDs can be targeted.

FIG. 2G depicts the DNA sequence-directed association of DST-f3 and DST-f4, followed by reconstitution of the split Ub moiety, the split Ub1 moiety, and r2. The fusion pair DST-f3 and DST-f4 are similar in overall design to the pair DST-f1/DST-f2 (FIGS. 2A, B, and D), but differ in that they contain a distinct pair of zinc fingers (ZF2-1 and ZF2-2) that recognize sequences in DNA-2, a segment of DNA whose sequence differs from that of DNA-1 (FIGS. 2E-G). If DNA-2 is present in a non-target cell, as shown here, an otherwise identical liberation of the reconstituted nuclease r2 takes place. In the embodiment depicted in FIGS. 2D-H, r2 has the same cleavage specificity as r1 but differs from r1 in being unable to cross-associate with the halves ofr1. Mechanistically, the association of DST-f3 and DST-f4 is similar to the association of DST-f1 and DST-f2, but the f3/f4 fusions recognize the presence of DNA-2, whereas f1/f2 recognize the presence of DNA-1. Because just one of these two association events (let alone both of them) would suffice for the activation of a restrictase(s) and destruction of DST vector (FIGS. 2E-G), any non-target cell, i.e., a cell containing at least one of two DNA segments, DNA-1 and/or DNA-2, would be spared.

Thus, in the depicted embodiment, the involvement of DNA-2 converts a DST circuit into a Boolean OR gate in that a DST vector would be destroyed if just one of two DNA segments, DNA-1 or DNA-2, is engaged by corresponding DST fusions (FIG. 2 D-G).

FIG. 2H provides a summary of one embodiment of events in a target cell (i.e., a cell comprising each of the targeted HDs, in this case, containing both HD1 and HD2 deletions) that received DST vector. In the embodiment depicted in FIG. 2H, neither DNA-1 nor DNA-2 is present in a target cell, owing to the deletions HD1 and HD2. As a result, the reconstitution of r1 and/or r2 restrictases, which require the binding of ZF domains ZF1-1/ZF1-2 to (nonexistent) DNA-1, and/or of ZF2-1/ZF2-2 to (nonexistent) DNA-2, does not take place, and the DST vector stays intact. After an (empirically optimized) time delay, to allow DST circuits to search for DNA-1 and/or DNA-2, the expression of the vector's payload P (FIG. 2C) can be activated.

In some embodiments, delayed induction of payload P is “manual,” i.e., it is controlled by the operator. In other embodiments of DST, the induction of payload P can be “automated” by, for example, making the induction a part of additional (time-delay) circuit encoded by the DST vector. In some embodiments, induction of the payload can be controlled by another vector. In preferred embodiments, the payload P-induction step can be manual, in order to, for example, control the step of the payload's activation from “outside.” In some embodiments, the state of the DST vector (intact or destroyed) is verified by independent tests in, for example, at least some cells of a patient. After verification of the state of the DST vector, an inducer of the payload's expression can be administered.

If the aim is to kill target cells, rather than to induce their terminal differentiation or label them, the range of possible proteins to serve as a payload of the DST vector is quite broad and includes, for example without limitation, bacterial and plant toxins, for example, diphtheria toxin or ricin. In other embodiments, the payload can include a proapoptotic protein, such as a caspase or a proapoptotic Bcl-2 family member such as, for example Bax or Bad. Expression of such toxins in a target cell would result in killing of the target cell. In some embodiment, payload P (FIG. 2C) can be a conditionally toxic protein. For example, the herpes simplex virus thymidine kinase (HSV-tk) can be used in combination with its substrate acyclovir (Ausubel et al. (2006) Current Protocols in Molecular Biology (Wiley, New York,)). For the conditionally toxic proteins, the expression of the protein itself alone would not be enough to kill target cells, but the conditionally toxic protein in combination with one or more agents is toxic. Other options include, for instance, the use of small-compound dimerizers (Bayle et al. (2006) Chem Biol 13, 99-107; Spencer et al. (1993) Science 262, 1019-1024), in which case the payload P (FIG. 2C) can be expressed as a split (conditional) toxin, with domains that bind to a cell-penetrating dimerizer, bringing the two halves of a toxic protein together in the presence of dimerizer and thereby making it possible to uncouple the induction of the payload's expression from the step of actually killing a target cell.

In other embodiments, the payload P can be a biomarker, such as, for example without limitation, green fluorescent protein (GFP).

A gene that encodes payload P can be controlled, for example, by a nonleaky inducible promoter. In some embodiments, the nonleaky inducible promoter can be activated by a small-compound inducer, such as, for instance, doxycycline or ecdysone (Schonig et al. (2002) Nucleic Acids Res 30, e134; Saez et al. (2000) Proc Natl Acad Sci USA 97, 14512-14517). Inducible promoters are known in the art and include, for example, chemically-regulated promoters and physically-regulated promoters (e.g., light and temperature sensitive promoters).

As mentioned in foregoing descriptions, the delivery of DST vector into cells can be nonspecific, because the selectivity of DST is an intracellular effect. Preferably, a nonspecific, high-efficiency delivery method is used, so that a sizable fraction of patient's cells, without regard to their nature, locations, or cell-cycle positions, receive a (transient) visit by DST vector in a given round of DST therapy. This would maximize the probability of not missing any target cells, irrespective of their heterogeneity in surface properties and other traits. A more compact summary of the specific DST circuit embodiment depicted in FIG. 2 and its mode of operation is provided in FIGS. 3A-B. FIG. 3A shows a flow chart of one embodiment of DST in normal (non-target) cells, which contain either both DNA-1 and DNA-2 segments or at least one of them. All of such cells are spared, given the logic of DST circuits described herein. FIG. 3B shows a flowchart of one embodiment of DST in target cells, lacking both DNA-1 and DNA-2 segments. Of course, specific delivery methods could also be used where available if applicable to the desired use.

### Homozygous DNA Deletions

The HDs that are chosen for targeting are the selectivity determinants of the DST strategy, and the methods disclosed herein. The operation of the DST circuit, including the final stage at which the “decision” is made to either destroy a DST vector (if it entered a normal cell) or to allow the activation of a vector's payload (e.g., a cytotoxic protein) is described, step by step, above. Given the logic of DST, second, third, fourth or additional HDs can be added as concurrent targets. Because the negative “spare this cell” output of a DST circuit is a part of its operation as a Boolean OR gate, an incremental addition of HDs as targets would increase the selectivity of treatment exponentially rather than linearly. Thus, the DST circuit can be designed to activate the payload only in cells having all of the specified HDs targeted by the fusions encoded by the DST vector; if a single targeted HD is missing from a given cell, the DST vector is destroyed.

Deletions that are relevant to the DST strategy are those that are at least partially homozygous and involve unique nucleotide sequences. Besides their advantage of zero reversion frequency, an HD is also a “digital” entity, in that the absence versus presence of a DNA sequence enables more robust designs that use deletions as targets.

The flexibility of DST in regard to repeated treatments has yet another advantage: it is unnecessary to focus on the same set of homozygous deletions throughout a DST therapy. For example, in some embodiments, a first treatment can include targeting a first set of HDs. Subsequently, a treatment to a second set of HDs can be administered. Additional treatments targeting additional HDs can be carried out if necessary.

Although FIG. 1B depicts HD1 and HD2 as being on separate chromosomes, this is not an actual constraint, because a set of relevant HDs can be present, a priori, anywhere in the genome. Cancer-associated HDs are presumed to form early in the process that led to a specific cancer, in part because some HDs eliminate genes for tumor suppressors. However, the known cancer-associated HDs (Finnis et al. (2005) Hum Mol Genet 14, 1341-1349; Kost-Alimova et al. (2007) Sem Cancer Biol 17, 19-30; Modena et al. (2005) Cancer Res 65, 4012-4019; Tagawa et al. (2005) Oncogene 24, 1348-1358; Jonsson et al. (2007) Genes Chromosomes Cancer 46, 543-558; Sun et al. (2007) Prostate 67, 692-700; Nakaya et al. (2007) Oncogene 26, 1-9; Cox et al. (2005) Proc Natl Acad Sci USA 102, 4542-4547; Struski et al. (2007) Cancer Genet Cytogenet 174, 151-160; Hamaguchi et al. (2002) Proc Natl Acad Sci USA 99, 13647-13652; Hustinx et al. (2005) Cancer Biol Ther 4, 83-86; Kasahara et al. (2006) Anticancer Res 26, 4299-4306; Seng et al. (2005) Genes Chromosomes Cancer 43, 181-193; Largo et al. (2007) Haematologica 92, 795-802) were identified either in cell lines established from tumors or by examining advanced cancers. Therefore it is still unclear whether a given cancer-associated HD is present in all cancer cells of a patient or in a large subset of them. Because the choice of HD targets remains flexible throughout DST therapy, the above (potential) complication can be dealt with by altering, if necessary, a set of targeted HDs. Again, as discussed above, multiple rounds of DST therapy can be carried out, each time targeting a different set of HDs.

In some embodiments, an HD can result in the complete or partial deletion of a known recessive cancer gene, a fragile site, or other location including, for example without limitation, CDKN2A/p16, PTEN, hMAD4, RB1, SMARCB1, FRA2G, FRA3B, FRA6E, FRA6F, FRA7G, FRA7H, FRA9E, FRA16D, and FRAXB. Many HDs are known, and additional HDs be identified using known in the art. See, for example, Cox et al., (2005) 102(12):4542-4547 and accompanying online supporting information.

In some embodiments, an alteration in a set of targeted HDs can illuminate the temporal position of specific HDs in the history of a cancer. For example, if a choice of specific HDs as DST targets leads to eradication of cancer cells in a patient, this result would suggest that the chosen HDs were present in the earliest population of tumorigenic cells that gave rise to that cancer.

In some embodiments, known HDs that are relevant for a particular cancer type can be targeted. In other embodiments, HDs are identified in cancer cells from a patient, and the identified HDs are targeted using the DST strategy. In some embodiments, HDs that are not associated with a specific disorder, but identify a population of cells in which expression of a particular payload is desired, can be targeted.

### Treatment of Cancer

As discussed above, many cancers harbor HDs. In contrast to other attributes of cancer cells, their HDs are immutable features that typically do not change during tumor progression or therapy. Germline DNA of phenotypically normal humans has been shown to contain, on average, about 30 hemizygous deletions larger than 5 kb, with regions of hemizygocity owing to deletions encompassing about 550 kb altogether (<0.02% of the genome) (Conrad et al. (2006) Nat Genet 38, 75-81; Sebat et al. (2004) Science 305, 525-528). Given the rarity of these hemizygous deletions, the bulk of homozygous deletions observed in cancer cells are de novo ones, acquired during tumor initiation and/or progression. Cancer-associated deletions that are relevant to the DST strategy are those that are at least partially homozygous and involve unique nucleotide sequences.

A DST system verifies the physical absence of a (homozygously) deleted DNA. In doing so, this circuit operates as a Boolean OR gate, erring on the side of caution: If the DST system (FIGS. 2 and 3) appears to detect even one (of two or more) segment of DNA that is supposed to be absent from a target cell, the DST vector's genome is designed to irreversibly self-destruct, and the cell is spared. This happens before the cytotoxic step (the activation of the vector's payload) is even “considered” by a circuit. Depending on the specifics of a DST regimen, such an extent of double-checking may result, stochastically, in letting go of some target cells that contain one or more HDs. However, if the therapy's selectivity for target cells versus non-target (normal) cells is high enough, the resulting disappearance of side effects brings forth an opportunity that other, less-selective therapies are less able to afford: the option of repeated treatments. The nonreversion property of homozygous deletions is synergistic with the possibility of making the frequency of DST's error (i.e., the error of killing non-target cells) arbitrarily low. As a result, a DST treatment can be administered repeatedly, with the usual concerns about side effects or alterations of targets either diminished or nonexistent.

Some embodiments disclosed herein provide methods of treating cancer in an individual by administering to an individual having cancer a DST vector as disclosed herein to target one or more HDs known to be present in the cancer. The term “cancer” is intended to mean a class of diseases characterized by the uncontrolled growth of aberrant cells, including all known cancers, and neoplastic conditions, whether characterized as malignant, benign, soft tissue or solid tumor. Specific cancers include, without limitation, digestive and gastrointestinal cancers, such as anal cancer, bile duct cancer, gastrointestinal carcinoid tumor, colon cancer, esophageal cancer, gallbladder cancer, liver cancer, pancreatic cancer, rectal cancer, appendix cancer, small intestine cancer and stomach (gastric) cancer; breast cancer; ovarian cancer; lung cancer; renal cancer; central nervous system (CNS) cancer, including brain cancer; prostate cancer; hematopoietic neoplasms such as leukemia, lymphoma and melanoma; skin cancers, eye cancers, and the like. Any cancer for which one or more HDs can be identified in can be treated using the methods disclosed herein.

In some embodiments, methods for treating cancer by killing cancer cells in an afflicted individual are provided. Accordingly, a nucleic acid vector for targeting one or more HDs known to be present in the target cancer cells is administered to a patient in need of such treatment. A therapeutically effective amount of the vector can be administered as a composition in combination with a pharmaceutical vehicle.

A therapeutically effective amount of a nucleic acid DST vector for targeting one or more HDs (DST vector), when used to treat cancer, is an amount required to allow killing in a target cell when an inducing agent is administered to an individual. The dosage of a DST vector required to be therapeutically effective will depend, for example, on the cancer to be treated, the payload expression inducing agent, the route and form of administration, the weight and condition of the individual, and previous or concurrent therapies. The appropriate amount considered to be an effective dose for a particular application of the method can be determined by those skilled in the art using the guidance provided herein and well known methods. For example, the amount can be extrapolated from in vitro or in vivo assays. One skilled in the art will recognize that the condition of the patient can be monitored throughout the course of therapy and that the amount of the vector that is administered can be adjusted accordingly.

By virtue of the cytopathic effect on individual cells, in some embodiments the disclosed methods can reduce or substantially eliminate the number of cells added to a tumor mass over time. Preferably, various methods disclosed herein effect a reduction in the number of cells within a tumor, and, most preferably, the method leads to the partial or complete destruction of the tumor (e.g., via killing a portion or substantially all of the cells within the tumor).

Those skilled in the art will know how to determine efficacy or amounts of a DST vector to administer. This can be based, at least in part, on the results of routine tests in a relevant animal model. The amount of a vector and inducing agent to be administered can be determined in a clinical setting as well based on the response in a treated individual. Modulation of efficacy will depend on the cancer and the extent to which progression of cell death is desired for treatment or reduction in the severity of the cancer. Modulation can be accomplished by adjusting the particular vector, inducing agent, formulation, or dosing strategy. Based on the guidance provided herein and what is well known in the art, those skilled in the art will be able to modulate efficacy in response to well known indicators of the severity of the particular condition being treated. For a description of indicators for the various cancers described herein see, for example, The Merck Manual, Sixteenth Ed, (Berkow, R., Editor) Rahway, N.J., 1992.

In one embodiment, HDs are identified in a cancer patient. A DST vector including ORFs for fusions for targetting one or more HDs in the cancer cells is prepared. The DST vector also includes an ORF for a payload, e.g., a toxin, under the control of an inducible promoter. The DST vector is administered to the cancer patent. Sufficient time for expression of the fusions and inactivation of the DST vector in non-cancer cells is allowed. The status of the DST vector (intact or cleaved) is verified in a sample of cells from the patient. After verification that the DST vector has been inactivated in non-cancer cells, an agent for inducing expression of the payload is administered to the patient. The payload toxin is expressed in the cancer cells, thereby killing the cancer cells.

### Ex Vivo Applications

Methods, structures, vectors and compositions disclosed herein, for example, DST vectors that target an HD, can be used in ex vivo applications, for example, purification or labeling of cell samples. In some embodiments, the methods, structures, vectors and compositions disclosed herein can be used in ex vivo methods to identify or kill cells having an HD. In many applications, it can be desirable to remove cancerous or precancerous cells from a population of cells. Exemplary ex vivo applications of the methods, vectors, fusions and compositions disclosed herein include, but are not limited to, blood banking; in vitro fertilization, for example, egg preservation or sperm preservation, both for human and veterinary applications; stem cell based products, including embryonic, fetal and adult stem cells; hybridomas for monoclonal antibody production; genetically engineered cells producing recombinant proteins; skin grafts; organ preservation for allograft and transplantation, and the like. Various embodiments thus additionally provide methods of purifying cell populations using the methods, structures, vectors and compositions disclosed herein.

In other embodiments, methods are provided for reducing the ability of a cell to survive ex vivo. The methods can include the steps of introducing into a cell ex vivo a vector encoding protein fusions and a payload. The cell can be contacted with the vector and inducing agent using the methods described herein for killing a cell having an HD. The methods can be used to kill and thereby remove a particular subpopulation of cells in a sample containing a population of cells using the targeting methods described herein.

In one embodiment, HDs are identified in target cells. A DST vector including ORFs for fusions for targeting one or more HDs in the target cells is prepared. The DST vector also includes an ORF for a payload, e.g., GFP, under the control of an inducible promoter. The DST vector is administered to a population of cells ex vivo. Sufficient time for expression of the fusions and cleavage of the DST vector in non-target cells is allowed. The status of the DST vector (intact or cleaved) is verified in at least a subset of cells in the sample. After verification that the DST vector has been inactivated in non-target cells, an agent for inducing expression of the payload is administered to the sample. The payload is expressed in the target cells, thereby labeling the target cells with GFP and allowing for their identification in the sample. In other embodiments the payload is a toxin and target cells within the population of cells are killed.

### Variations

Although the above example describes DST in the context of homozygous deletions (FIGS. 1-3), a DST-type strategy is also relevant to any setting in which one wishes to distinguish amongst sets of cells that contain or lack specific DNA sequences and to target one or the other such set. Thus, cell populations with specific missense mutations, chromosomal translocations, hemizygous deletions (as distinguished from HDs), and copy-number increases in specific DNA regions (gene amplification) can also be a part of DST-type strategies. A major difference between DST-relevant homozygous deletions and other genetic alterations are both the permanence of HDs and their “digital” (all-or-none) quality, accentuated by relatively large sizes of cancer-associated HDs: hence the focus on homozygous deletions.

Although HDs themselves would not cause increased resistance to treatment, it may be possible that other sources of “acquired” resistance can occur. However, they can be dealt with, because repeated treatments are feasible. The DST strategy is based on macromolecular structures, and involves the entrance of vectors encoding them into cells. Thus, it is possible that a resistance to DST treatment can build up in ways that are similar to the routes that increase resistance to other cancer therapies as well. For example, if a DST vector is delivered into cells using a virus or a liposome-encapsulated DNA, the changing genetic landscape of a patient's cancer (in part because previous rounds of DST therapy eliminated a subset of cancer cells) may result in the remaining cancer cells being more resistant to entry of DST's carrier. A remedy, given the possibility of repeated DST treatments, would be to retain the DST vector but to deliver it through a different virus or modified liposomes. Strategies of this kind would also be expected to deal with the problem of a patient's immune responses to a viral vector.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Activation of a Payload in a Cell Having a Homozygous Deletion

This example illustrates one embodiment of the activation of a payload in a target cell having a homozygous DNA deletion (HD).

An HD is identified in a target cell. The sequence of a DNA segment that has been removed as a result of the HD is determined.

A DST vector for targeting the HD is prepared. The DST vector includes ORFs for two complementary fusions that recognize the deleted DNA segment. Expression of the fusions is regulated by the constitutive P_{CMV} promoter. The fusions each contain a DNA binding domain that recognizes a sequence in the deleted DNA segment. The recognition sequences of the complementary fusions are adjacent, and binding of the complementary fusions to their adjacent recognition sequences brings them in close enough proximity that they are able to reconstitute a site-specific endonuclease. One fusion includes an N terminal fragment of a site-specific endonuclease, and the other fusion includes a C terminal fragment of the site-specific endonuclease. The fragments of the site-specific endonuclease are modified such that moderate coexpression does not reconstitute endonuclease activity, but if the fragments are brought into close spatial proximity, endonuclease activity is reconstituted. The binding of the fusions to their recognition sequences in cells lacking the targeted HD brings together the C terminal and N terminal fragments of the site-specific endonuclease and reconstitutes endonuclease activity. The DST vector includes multiple cleavage sites for the endonuclease. Each of the fusions also includes two “releasing domains” between the DNA binding domain and endonuclease fragment. The releasing domains facilitate release of the reconstituted endonuclease. The releasing domains can include, for example, split Ub and split Ub1. The binding of the fusions to their recognition sequences in cells lacking the targeted HD brings together two halves of Ub and Ub1 and reconstitutes the activities of the Ub and Ub1. The DST vector also encodes a payload protein, GFP. Expression of GFP is regulated by the P_{tetO} promoter.

The DST vector is administered to a population of cells including at least one cell having the targeted HD (a target cell). Administration of the DST vector can be carried out by any suitable means, including using liposomes, viruses, chemical transfection or any other suitable means. After a suitable time for expression and binding of the fusions, and cleavage of the DST vector in cells lacking the targeted HD, tetracycline for inducing the inducible promoter is administered to the population of cells. GFP is expressed only in cells having the targeted HD. In cells lacking the targeted HD, the DST vector is destroyed, and GFP is not expressed. The population of cells can be examined for GFP expression using, for example, UV light.

### Example 2

### Activation of a Payload in a Cell Having Two Homozygous Deletions

This example illustrates one embodiment of the activation of a payload in a target cell having two homozygous DNA deletions, HD1 and HD2.

Two HDs, HD1 and HD2, are identified in a target cell. The sequence of a DNA segment, DNA-1, which has been removed from wild type cells as a result of HD1 is determined. The sequence of a DNA segment, DNA-2, which has been removed from wild type cells as a result of HD2 is determined. A DST vector for targeting the HDs is prepared. The DST vector includes an ORF for DST-f1, which includes a DNA binding domain (ZF1-1) which recognizes a 9-bp (or greater) sequence of DNA-1. The DST vector also includes an ORF for DST-f2, which includes a DNA binding domain which recognizes a 9-bp (or greater) sequence of DNA-1 adjacent to the 9-base (or greater) pair recognition sequence of ZF1-1. DST-f1 and DST-f2 further includes split restrictase, split-Ub and split-Ub1 domains as described above. The DST vector also includes ORFs for complementary protein fusion molecules DST-f2 and DST-f3, which include DNA binding domains which recognize adjacent recognition sequences within DNA-2. In addition, the DST vector includes an ORF for a payload, a cytotoxic protein. Payload expression, and thus activation, is under the control of an inducible promoter.

The DST vector is administered to a population of cells including at least one cell having the both HD1 and HD2 (a target cell). Administration of the DST vector can be carried out by any suitable means, including using liposomes, viruses, or any other suitable means. After a suitable time for expression and binding of DST-f1, DST-f2, DST-f3 and DST-4, and cleavage of the DST vector in cells lacking HD1 or HD2, or both HD1 and HD2, an agent for inducing the inducible promoter is administered to the population of cells. In some embodiments, the agent can be added after about 24 to about 48 hours after administration of the DST vector. The cytotoxic protein is expressed only in target cells, and the target cells are killed. In cells lacking one or both HDs, the DST vector is destroyed, and the payload is not activated.

### Example 3

### DST Therapy for Treatment of Cancer

This example illustrates one embodiment of treating a patient with cancer using DST.

A patient diagnosed with cancer is selected for treatment with DST. Homozygous DNA deletions HD1 and HD2 are identified in cancer cells of the patient, and sequence segments DNA-1 and DNA-2 are identified as DNA segments deleted by HD1 and HD2, respectively (DNA-1 is a segment of DNA deleted by HD1, and DNA-2 is a segment of DNA deleted by HD2). In this example, HD1 and HD2 are deletions of CDKN2A/p16, a known cancer related gene. Zinc finger (ZF) domains ZF1-1, ZF1-2, ZF2-1 and ZF2-2, which recognize adjacent sequences in DNA-1 and DNA-2 are identified (ZF1-1 and ZF1-2 recognize adjacent sequences in DNA-1, and ZF2-1 and ZF2-2 recognize adjacent sequences in DNA-2).

A DST vector for targeting HD1 and HD2 is prepared. The DST vector includes ORFs for four protein fusion molecules: two complementary fusions that recognize DNA-1 (DST-f1 and DST-f2), and two complementary fusions that recognize DNA-2 (DST-f3 and DST-f4). DST-f1, DST-f2, DST-f3 and DST-f4 include the ZF domains ZF1-1, ZF1-2, ZF2-1 and ZF2-2, respectively. Expression of the fusions is regulated by the constitutive P_{CMV} promoter. Each fusion also includes an N or C terminal fragment of yeast SceI endonuclease. The fragments of SceI are modified such that moderate coexpression does not reconstitute SceI activity, but if the fragments are brought into close spatial proximity, SceI activity is reconstituted. The binding of DST-f1 and DST-f2 to their recognition sequences in cells lacking HD1 brings together two halves of SceI and reconstitutes the activity of SceI. Similarly, the binding of DST-f3 and DST-f4 to their recognition sequences in cells lacking HD2 brings together two halves of SceI and reconstitutes the activity of SceI. The DST vector includes multiple cleavage sites for SceI. Each of the fusions also includes two “releasing domains” between the ZF domain and SceI fragment. The releasing domains facilitate release of the reconstituted SceI. The releasing domains can include, for example, split Ub and split Ub1. The binding of DST-f1 and DST-f2 to their recognition sequences in cells lacking HD1 brings together two halves of Ub and Ub1 and reconstitutes the activities of the Ub and Ub1. Similarly, binding of DST-f3 and DST-f4 to their recognition sequences in cells lacking HD2 brings together two halves of Ub and Ub1 and reconstitutes the activities of the Ub and Ub1. The DST vector also encodes a payload protein, ricin. Expression of ricin is regulated by the P_{tetO}.

The patient is given a therapeutically effective intravenous dose of the DST vector. A suitable time period is allowed for reconstitution of SceI activity and destruction of the DST vector in cells lacking one or both HDs. A sample from the patient is analyzed to verify the status of the DST vector. After destruction of the DST vector in non-cancer cells has been verified, doxycycline, an agent for inducing expression of the payload protein, is administered to the patient. The cytotoxic protein is expressed in cancer cells only, killing the cancer cells.

Administration of the DST vector and doxycycline is repeated at regular intervals, allowing time for the inducing agent to clear from cells prior to each subsequent administration of DST vector. At three, six, nine, and twelve weeks of treatment, the patient is evaluated by for shrinkage, growth or metastasis of the tumor. Following the end of the treatment period it is observed that the cancer has regressed.

### Example 4

### DST Therapy for Treatment of Cancer

This example illustrates one embodiment of treating a patient with cancer tumor using DST.

A patient diagnosed with cancer is selected for treatment with DST. The cancer is known to have HD1 and HD2. DST vector for targeting HD1 and HD2 is provided. The DST vector includes ORFs for fusions that recognize DNA-1 and DNA-2. DNA-1 is a segment of DNA deleted by HD1, and DNA-2 is a segment of DNA deleted by HD2. The DST vector includes an ORF for a payload comprising a cytotoxic protein. Expression of the payload is controlled by a non-leaky inducible promoter.

The patient is given a therapeutically effective intravenous dose of the DST vector complexed with liposomes. After a suitable time period to allow destruction of the DST vector in cells lacking one or both HDs, an agent for inducing expression of the DST vector payload is administered to the cells. Administration of DST vector and the inducing agent is repeated at regular intervals, allowing time for the inducing agent to clear from cells prior to each subsequent administration of DST vector. At three, six, nine, and twelve weeks of treatment, the patient is evaluated by for shrinkage, growth or metastasis of the tumor. Following the end of the treatment period it is observed that the cancer has regressed.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein.

### Incorporation by Reference

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

### EQUIVALENTS

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. A method for selectively expressing a payload protein in a target cell having a homozygous DNA deletion (HD), comprising:
administering, to a population of cells comprising the target cell, a nucleic acid vector encoding at least two complementary protein fusion molecules and a payload protein such that the payload protein is selectively expressed in the target cell having said HD.

2. The method of claim 1, wherein the target cell is a cancer cell.

3. The method of claim 1, wherein the payload protein is a toxic protein.

4. The method of claim 1, wherein the payload protein is not expressed in cells lacking said HD.

5. The method of claim 1, wherein the target cell comprises two or more HDs, and the nucleic acid vector encodes:
at least four protein fusion molecules for targeting a set of said HDs; and
a payload protein such that the payload protein is selectively expressed in the target cell having said set, and the payload protein is not expressed in cells lacking one or more HDs of said set.

6. The method of claim 1, wherein said nucleic acid vector comprises:
a first nucleic acid sequence encoding a first protein fusion molecule comprising a first DNA binding domain and a first portion of a site-specific endonuclease; and
a second nucleic acid sequence encoding a second protein fusion molecule comprising a second DNA binding domain and a second portion of said site-specific endonuclease, wherein said first and second DNA binding domains recognize adjacent DNA sequences that are not present in the target cell, and wherein upon binding of said first and second DNA binding domains to said adjacent DNA sequences, said first and second protein fusion molecules form and release an active site-specific endonuclease.

7. The method of claim 6, wherein said nucleic acid vector further comprises:
a third nucleic acid sequence encoding said payload protein operably linked to an inducible promoter; and
at least one recognition site for said site-specific endonuclease.

8. The method of claim 7, further comprising:
expressing said first and second protein fusion molecules;
reconstituting activity of said site-specific endonuclease in non-target cells; and
administering to said population of cells an agent that induces expression of said payload protein in target cells.

9. A nucleic acid vector for selectively expressing a payload protein in a target cell having a homozygous DNA deletion (HD) comprising:
a first nucleic acid sequence encoding a first protein fusion molecule comprising a first DNA binding domain and a first portion of a site-specific endonuclease;
a second nucleic acid sequence encoding a second protein fusion molecule comprising a second DNA binding domain and a second portion of said site-specific endonuclease;
a third nucleic acid sequence encoding a payload protein; and
at least one recognition site for said site-specific endonuclease.

10. The vector of claim 9, wherein upon binding of said first and second DNA binding domains to adjacent DNA sequences, said first and second protein fusion molecules interact to form an active site-specific endonuclease.

11. The vector of claim 9, wherein said adjacent DNA sequences comprise sequences that are deleted by the HD.

12. The vector of claim 9, wherein said adjacent DNA sequences are spaced apart such that their binding surfaces are on the same side of the DNA helix.

13. The vector of claim 9, wherein said first DNA binding domain and said second DNA binding domain are each a zinc finger.

14. The vector of claim 9, wherein said site-specific endonuclease is a restriction endonuclease or a zinc finger nuclease.

15. The vector of claim 9, wherein said vector comprises multiple recognition sites for said site-specific endonuclease.

16. The vector of claim 9, wherein said first protein fusion molecule further comprises a first releasing domain, and said second protein fusion molecule further comprises a second releasing domain.

17. The vector of claim 16, wherein said first releasing domain comprises a C-terminal portion of ubiquitin (Ub) located between said first DNA binding domain and said first portion of a site-specific endonuclease, and said second releasing domain comprises an N-terminal portion of said Ub located between said second DNA binding domain and said second portion of said site-specific endonuclease.

18. The vector of claim 17, wherein said first protein fusion molecule further comprises a third releasing domain, and said second protein fusion molecule further comprises a fourth releasing domain.

19. The vector of claim 18, wherein said third releasing domain comprises an N-terminal portion of a ubiquitin-like protein (Ub1) located between said C-terminal portion of Ub and said first portion of a site-specific endonuclease, and said fourth releasing domain comprises a C-terminal portion of said Ub1 located between said N-terminal portion of said Ub and said second portion of said site-specific endonuclease.

20. The vector of claim 9, wherein said third nucleic acid sequence is operably linked to an inducible promoter.

21. The vector of claim 9, wherein the payload protein is a toxic protein.

22. The vector of claim 21, wherein the toxic protein is selected from the group consisting of a bacterial toxin and a plant toxin.

23. The vector of claim 21 wherein the toxic protein is herpes simplex virus thymidine kinase (HSV-tk).

24. The vector of claim 9, wherein the payload protein is a small compound dimerizer.

25. The vector of claim 9, wherein said nucleic acid vector is a DNA vector.

26. The vector of claim 9, wherein said target cell is a cancer cell.

27. A method of treating cancer in a patient, said method comprising:
administering to said patient a nucleic acid vector encoding two complementary protein fusion molecules and a payload protein, wherein the payload protein is selectively activated in cancer cells having a homozygous DNA deletion (HD).

28. The method of claim 27, wherein the nucleic acid vector is inactivated in cells that do not have said HD.

29. The method of claim 27, wherein said payload protein induces terminal differentiation of said cancer cells.

30. The method of claim 27, wherein said payload protein kills said cancer cells.

31. The method of claim 27, further comprising identifying said HD in cancer cells of a patient.

32. The method of claim 27, wherein said nucleic acid vector comprises:
a first nucleic acid sequence encoding a first protein fusion molecule comprising a first DNA binding domain specific and a first portion of a site-specific endonuclease;
a second nucleic acid sequence encoding a second protein fusion molecule comprising a second DNA binding domain and a second portion of said site-specific endonuclease, wherein said first and second DNA binding domains recognize adjacent DNA sequences, wherein said adjacent DNA sequences are deleted by said HD, and whereupon binding of said first and second DNA binding domains to said adjacent DNA sequences, said first and second protein fusion molecules can interact to form an active site-specific endonuclease;
a third nucleic acid sequence encoding a payload protein operably linked to an inducible promoter; and
at least one recognition site for said site-specific endonuclease.

33. The method of claim 32, further comprising expressing said first and second protein fusion molecules in non-cancer cells of said patient.

34. The method of claim 32, further comprising reconstituting activity of said site-specific endonuclease in non-cancer cells of said patient.

35. The method of claim 27, further comprising verifying the status of said nucleic acid vector in a sample of cells from said patient.

36. The method of claim 27, further comprising administering to said patient an agent that induces expression of said payload protein in cancer cells of said patient.
